(19) United States
(12) Patent Application Publication
(10) Pub. No.: US 2011/0054635 A1
(43) Pub. Date: Mar. 3, 2011

(54) PROSTHESIS AND RESIDUAL LIMB COVER

(76) Inventor: Robert John Watts, Hampshire (GB)

(21) Appl. No.: 12/733,945

(22) PCT Filed: Sep. 29, 2008
(86) PCT No.: PCT/EP2008/063028

§ 371 Date: Oct. 28, 2010

(30) Foreign Application Priority Data
Sep. 27, 2007 (GB) 0718863.4
May 13, 2008 (GB) 0808659.7

(51) Int. Cl. A61F 2/80; A61F 2/78; B29C 33/40

(52) U.S. Cl. 623/36; 623/33; 264/222

(57) ABSTRACT

This application relates to a prosthesis 12 and a cover 1 for a residual limb, wherein: the cover 1 comprises a first region 8 that is normally of a greater circumference than an adjacent second region of the cover, said first region being resiliently deformable so as to temporarily reduce the circumference of that region of the cover, and the prosthesis 12 comprises a portion that defines an internal cavity 11 into which a residual limb that is at least partly covered by said cover 1 may be inserted, wherein said cavity 11 is configured to include a first region 13 that has a first cross-sectional area and below said first region in the direction of limb insertion, a second region 15 that has a second cross-sectional area larger than said first, the arrangement being such that said resiliently deformable first region 8 of said cover deforms to reduce the circumference of that region of said cover as the cover 1 moves through said first region 13 of the cavity, and expands in said second region 15 of the cavity to thereby couple the prosthesis 12 to the user's covered residual limb. A cover with a nipple and a prosthesis with a complementary socket are also disclosed.

### FIELD OF THE INVENTION

One aspect of this invention relates to a cover for a residual limb. Another aspect of the invention relates to a prosthesis, and yet another aspect of the invention relates to a prosthesis and a residual limb cover that cooperate with one another to facilitate attachment of the prosthesis to a user.

In the context of the following description a residual limb includes a residual part of a leg and or arm as well as a hand or foot that is missing at least part of one or more digits or toes respectively. References hereafter to a residual limb should therefore be interpreted accordingly.

### BACKGROUND TO THE INVENTION

A variety of prostheses are known in the art. One illustrative example is an artificial leg of the type that might be fitted to a transfemoral amputee. This illustrative leg comprises an artificial foot that is coupled, via an artificial knee, to a socket that defines an internal cavity into which the user inserts their residual limb—in this example, the remaining portion of their upper leg.

It is commonplace for the user to cover their residual limb with a so-called “liner”, which liner functions to enhance the interface between the skin of the limb and the socket—in particular to improve comfort and stability for the user.

Artificial legs have heretofore been coupled to a residual limb by one of two mechanisms. The first of these mechanisms employs a vacuum to couple the prosthesis to the residual limb, and the second employs a locking bolt that extends from an attachment provided within the distal end of the liner to couple with a complementary fixing provided in the bottom of the cavity defined by the prosthesis socket.

An example of a vacuum attachment method is the Icecross® Seal-In liner and complementary socket. This liner carries, about its circumference in a region towards the distal end of the liner, a hypobaric sealing member (HSM) that cooperates with the internal wall of the socket as the user inserts their residual limb to drive air from the region of the socket cavity below the (HSM) out of a one-way valve provided in the socket to permit the escape of air to atmosphere. As the limb is pushed into the socket the air pressure within the region of the socket beneath the HSM reduces to couple the prosthesis to the limb. The valve incorporates a release mechanism that, once operated, functions to return the pressure within the cavity to atmospheric, whereupon the residual limb and cover can be withdrawn from the socket.

Whilst this arrangement functions adequately, it is a relatively complicated system that is rendered useless if the valve should become blocked or damaged (at least until the valve can be replaced). This is particularly inconvenient if the valve should be damaged once the cavity has been evacuated as removal of the residual limb will require disassembly of the prosthesis, and it may not be feasible for some users to attempt this without assistance. A further disadvantage is that this equipment can sometimes generate noise as the user walks or runs and air is repeatedly pushed out of the valve by the piston action of the liner moving slightly in the prosthesis. Another disadvantage that is particularly apparent when the user is sat down is that the vacuum attachment can cause the prosthesis to draw on the end of the residual limb. This drawing of the prosthesis on the residual limb can cause discomfort, often exemplified by a tingling sensation (so called “pins and needles”) in the distal end of the residual limb.

The aforementioned second method avoids such problems, but has its own set of disadvantages. A first of these is that as the distal end of the residual limb bears directly on the attachment (from which the locking bolt extends to mate with the fixing), the attachment can dig into the limb and cause considerable discomfort to the user. Another disadvantage is that the locking bolt and attachment add significantly to the length of the residual limb, and in some instances (for example in situations where the leg has been amputated just above the knee) the additional length added by the bolt and attachments cause the residual limb to be too long for use with certain prostheses.

It would be advantageous if an alternative mechanism could be devised for fixing a prosthesis to a residual limb.

### SUMMARY OF THE INVENTION

With this in mind, a presently preferred embodiment of the present invention provides a prosthesis and a cover for a residual limb, wherein: the cover comprises a first region that is normally of a greater circumference than an adjacent second region of the cover, said first region being resiliently deformable so as to temporarily reduce the circumference of that region of said cover, and said prosthesis comprises a portion that defines an internal cavity into which a residual limb that is at least partly covered by said cover may be inserted, wherein said cavity is configured to include a first region that has a first cross-sectional area and below said first region in the direction of limb insertion, a second region that has a second cross-sectional area larger than said first, the arrangement being such that said resiliently deformable first region of said cover deforms to reduce the circumference of that region of said cover as the cover moves through said first region of the cavity, and expands in said second region of the cavity to thereby couple the prosthesis to the user's covered residual limb. Another aspect of the present invention relates to a residual limb cover for a cooperating limb cover and prosthesis system, wherein said prosthesis comprises a portion that defines an internal cavity into which a residual limb that is at least partly covered by said cover may be inserted, said cavity being configured to include a first region that has a first cross-sectional area and below said first region in a direction of limb insertion, a second region that has a second cross-sectional area larger than said first, said cover comprising: a first region that is normally of a greater circumference than an adjacent second region of the cover, said first region being resiliently deformable so as to temporarily reduce the circumference of that region of said cover; the arrangement being such that said resiliently deformable first region of said cover is configured to deform to reduce the circumference of that region of said cover as the cover moves through said first region of the cavity, and to expand in said second region of the cavity to thereby couple the prosthesis to the user's covered residual limb.

Another aspect of the present invention relates to a prosthesis for a cooperating limb cover and prosthesis system, said limb cover comprising a first region that is normally of a greater circumference than an adjacent second region of the cover, said first region being resiliently deformable so as to temporarily reduce the circumference of that region of said cover; said prosthesis comprising: a portion that defines an internal cavity into which a residual limb that is at least partly covered by said cover may be inserted, said cavity being configured to include a first region that has a first cross-sectional area and below said first region in a direction of limb insertion, a second region that has a second cross-sectional area larger than said first, the arrangement being such that said resiliently deformable first region of said cover is configured to deform to reduce the circumference of that region of said cover as the cover moves through said first region of the cavity, and to expand in said second region of the cavity to thereby couple the prosthesis to the user's covered residual limb.

Another embodiment of the present invention relates to method of making a cover for a residual limb, the method comprising: forming a positive impression of the residual limb; forming a resiliently deformable pad on a distal end of said positive impression; and forming a skin on said resiliently deformable pad and at least a portion of the positive impression to provide said cover.

Preferred features of these embodiments are set out in the accompanying claims, and other features and embodiments of the present invention will be apparent from the following detailed description of preferred embodiments of the present invention.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is an illustrative representation of a limb cover according to a preferred embodiment of the invention;

FIG. 2 is a cross-sectional view through the cover of FIG. 1;

FIG. 3 is a schematic representation in cross-section of part of a prosthesis illustrating the manner in which the cover of FIGS. 1 and 2 co-operates with the prosthesis to couple the prosthesis to a user's residual limb;

FIG. 4 is a photograph depicting a limb cover according to another embodiment of the invention;

FIG. 5 is a cross-sectional view through the limb cover of FIG. 4;

FIG. 6 is a cross-sectional view through a limb cover according to another embodiment of the present invention; and

FIG. 7 is a schematic representation in cross-section of part of a prosthesis that cooperates with the cover depicted in FIGS. 3 to 6.

### DETAILED DESCRIPTION OF PREFERRED EMBODIMENTS

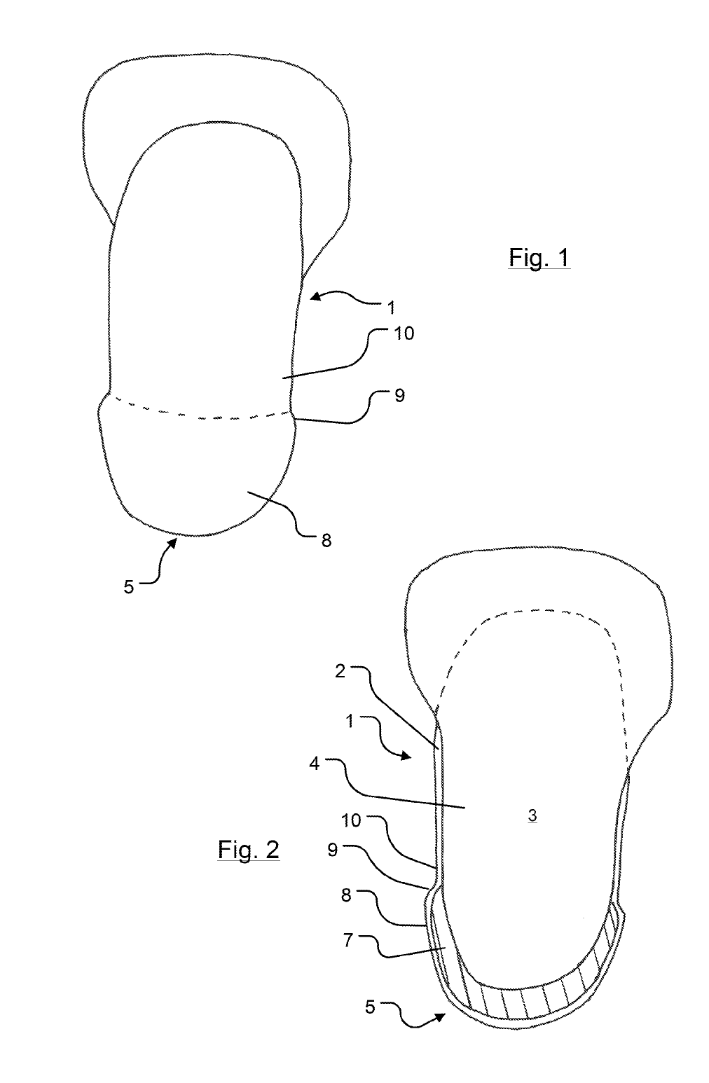

FIG. 1 is a schematic elevation of a cover 1 for a residual limb (not visible in FIG. 1). In this instance the residual limb comprises the upper part of a leg, but the teachings of the present invention are equally applicable to lower leg prostheses, upper and lower arm prosthesis, and to hand, foot, finger and toe prostheses.

Referring now to FIGS. 1 and 2 (where FIG. 2 is a cross-sectional view through the cover of FIG. 1), the cover 1 comprises a skin 2 that is formed from silicone, or other suitable resilient material, to define a void 3 for receiving a user's residual limb 4. A distal end 5 of the cover 1 includes an oversized resilient member 7 (which may for example be in the region of 5-15 mm, preferably around 7-10 mm, thick at its widest point), for example a gel pad of silicone or other suitable resiliently compressible material, that forms a region 8 that has a circumference that is larger than an adjacent region 10 of the cover 1, the transition from region 10 to region 8 being defined by a lip 9 that extends around the circumference of the cover. The skin is typically in the region of 1 to 5 mm, preferably roughly 2 mm thick, and may have a shore hardness of 10 to 40, preferably a shore hardness of 10 to 30, most preferably a shore hardness of roughly 20. In a particularly preferred embodiment the skin is of silicone elastomer, for example Med-4014™ silicone elastomer (or equivalent) available from NuSil Technology LLC, 1050 Cindy Lane, Carpinteria, Calif. 93013, USA.

The region 8 of larger circumference is typically, for an adult user, no larger than 20 mm greater in diameter than the smallest diameter of the adjacent region 10, and typically between 10 and 20 mm greater in diameter than the smallest diameter of the adjacent region 10. For children and smaller adults, the differential between the diameters of regions 8 and 10 is likely to be smaller.

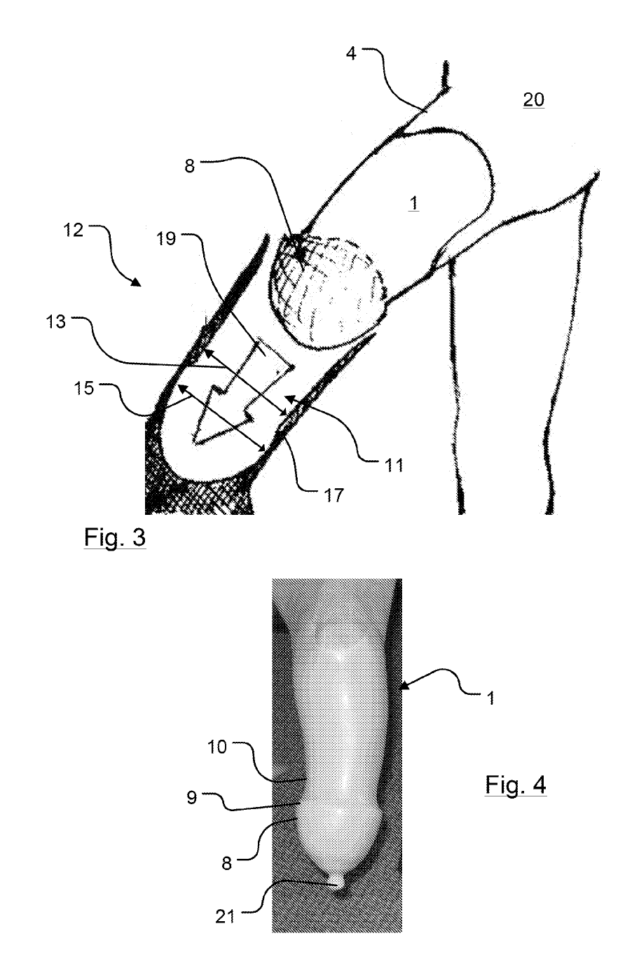

FIG. 3 is a cross-sectional view of a socket part 12 of a prosthesis. The socket part 12 defines an internal void 11 that includes a first region 13 with a first cross-sectional area. The void 11 further comprises a second region 15 beyond a lip 17 that has a second cross-sectional area which is larger than the first region 13. As shown, the second region 15 is located beneath the first region (i.e. closer to the foot, in this instance) in a direction 19 of residual limb insertion.

In use, a user 20 puts the cover 1 onto their residual limb 4 and pushes the residual limb into the void 11 of the socket part 12. As the limb and cover move into the void 11, cover region 8 bears against socket region 13 and temporarily deforms (squashes) in a generally inward direction to reduce the circumference of cover region 8. As cover region 8 moves beyond the lip 17 it expands to its original size and cover lip 9 cooperates with socket lip 17 to couple the cover (and hence the residual limb over which the cover is received) to the prosthesis.

To remove the limb and cover from the prosthesis the user need only pull downwards on the prosthesis whereupon cover region 8 deforms once more in a generally inward direction until cover lip 9 can pass by socket lip 17 and the limb can be removed.

As will be appreciated by persons skilled in the art, the ease with which the residual limb and cover can be removed depends to a large extent on how sharp a change in circumference is provided by lips 9 and 17. In particular, as the change in circumference per unit distance increases (i.e. as lips 9 and 17 become steeper) so the ease with which the limb and cover may be withdrawn decreases. Another factor affecting the ease with which the residual limb and cover can be withdrawn is the resilience of the resilient member. In particular, the greater the resilience of the resilient member, the harder it is to squash the member and hence the harder it is to remove the limb and cover from the prosthesis. A surprising technical advantage of the proposed arrangement is that the resilient member can be configured to have a resilience which is both sufficiently low to enable the enlarged region of the cover to deform as the limb is inserted into the prosthesis, and sufficiently high to resist removal of the covered limb from the prosthesis in use. Another advantage is that as the resilient member can deform when a user sits down, the extent to which the prosthesis draws on the distal end of the residual limb is reduced (at least in comparison to a vacuum attachment system of the type aforementioned).

The degree of resilience required will of course depend on the user and the use to which they intend to put the cover and prosthesis, but in general terms the resilience of the resilient member may be from 20 to 40 shore hardness, preferably from 25 to 35 shore hardness, and most preferably roughly 30 shore hardness.

In a particularly preferred embodiment the resilient member comprises a silicone gel pad, such as a gel pad formed of Ecoflex™ silicone elastomer available from Smooth-On Inc., 2000 St. John Street, Easton Pa. 18042, USA.

FIG. 4 is a photograph of a limb cover according to a second embodiment of the present invention.

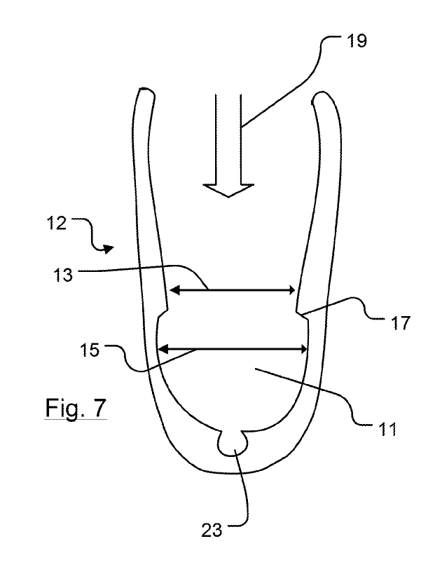

The limb cover of this embodiment is similar to that of FIG. 1, the difference being the provision of a resiliently deformable nipple 21 that extends from a distal end of the cover. The nipple deforms to fit within a complementary socket 23 (shown in FIG. 7) formed in the distal end of the prosthesis when the user inserts their covered limb into the prosthesis. The engagement of the nipple with the socket provides an additional attachment point between the cover and the prosthesis to enhance the coupling between the cover and the prosthesis, and our studies have found that this increase in coupling force is particularly valuable for those users who are intending to undertake more vigorous activities, such as jogging for example. For those users who are not intending to undertake more vigorous activities, the arrangement depicted in FIG. 1 is adequate to couple the cover to the prosthesis.

Referring now to FIG. 5, in which there is shown a schematic cross-sectional view through the limb cover of FIG. 4, in one embodiment of the present invention the nipple 21 is formed integrally with the skin of the cover.

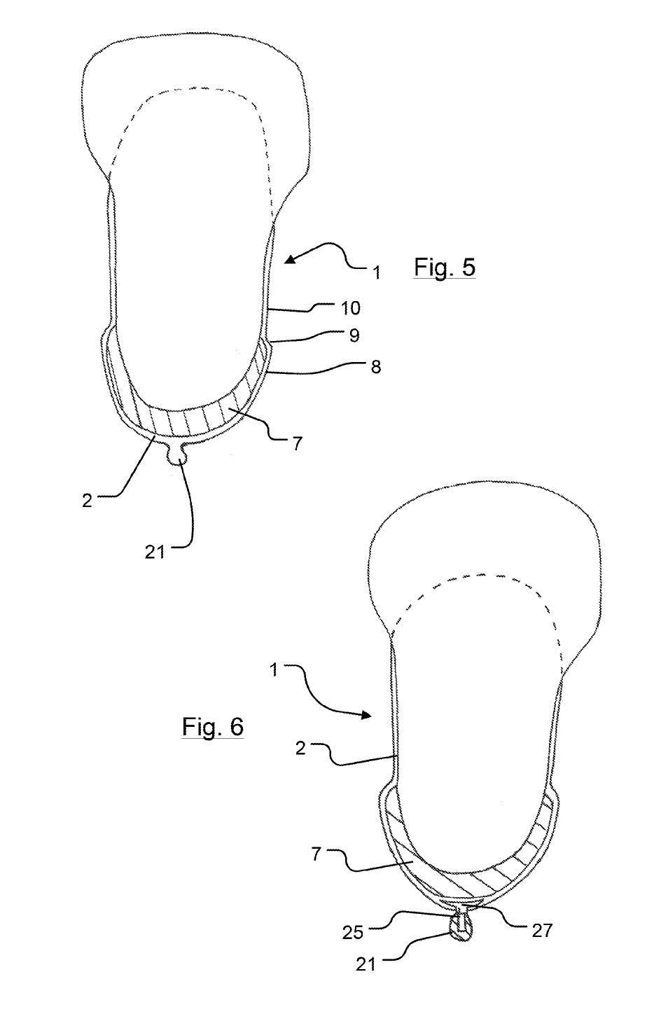

In another embodiment shown schematically in FIG. 6, the nipple 21 may be separable from the remainder of the cover. In one arrangement, the nipple 21 may be formed on a peg 25 that can be fitted into a retaining member 27 encapsulated within the cover (preferably with the reslient member 7 between the retaining member 27 and the user's residual limb). In an illustrative arrangement the peg may be externally threaded and the retaining member may include an internally threaded bore that the threaded peg can be screwed into. In another arrangement, the peg may include a deformable circumferential ring and the bore may include an internal region of larger circumference into which the ring may resilient expand when the peg is inserted into the bore.

One advantage of this arrangement is that the nipple can be removed from the cover when the user is not likely to undertake more vigorous activities. Another advantage is that the nipple can be replaced if it should be damaged. For some patients the engagement of the nipple with the socket may cause the prosthesis to draw on the residual limb in certain circumstances, for example when the user is seated, and to avoid this problem the socket can be provided with a releasable locking mechanism so that the nipple can be released from the socket, for example when the user is seated. A variety of suitable locking mechanisms are known in the art. For example, the socket may take the form of or include a so-called shuttle lock that is capable of securely receiving the nipple, and can be released (for example by means of a button on the side of the prosthesis) to release the nipple from the socket. With such an arrangement a user can release the nipple from the socket when seated, and upon standing once more the nipple will automatically enter and engage with the socket. In an alternative arrangement, the nipple may be replaced with a pin that extends distally from the retaining member (and may be removable from the retaining member), and the socket may be provided with a ratchet lock mechanism that engages with the pin. Such pin and ratchet locking mechanism are well known in the art and provide the user with the ability to slacken the coupling between the pin and the ratchet lock to reduce draw on the residual limb.

As aforementioned, in a particularly preferred embodiment of the present invention the resilient member comprises a gel pad and both the resilient member and the skin are of silicone elastomer.

A cover having the features described herein may be manufactured by any of a number of different methods, and in one illustrative embodiment the cover is formed on a positive impression of the user's residual limb.

In general terms, this method comprises the steps of forming a positive impression of the residual limb; forming a resiliently deformable pad on a distal end of said positive impression; and forming a skin on said resiliently deformable pad and at least a portion of the positive impression to provide said cover.

In more detail, in a first step of this method a negative impression is taken of the user's residual limb by covering the limb with, for example, plaster of paris. The user's residual limb is withdrawn once the plaster of paris has cured and a positive impression of the residual limb is then formed using the negative impression as a mould. Lengths of silicone elastomer are then affixed to the distal end of the positive impression to form an appropriately shaped enlarged cover portion that has a void between the silicone and the positive impression of the user's residual limb. The silicone includes a tail that forms a feed tube (to provide access to the void) when additional plaster of paris is applied to the shaped silicone to form a negative enlarged portion mould having a shape corresponding to that of the shaped silicone. The silicone is then removed from the enlarged portion mould, the enlarged portion mould is reaffixed to the positive impression of the residual limb and, in the preferred embodiment, silicone gel, is fed into the void via the feed tube to form the resilient member. When the resilient member has cured, the negative enlarged portion mould is removed and the enlarged portion and the remainder of the positive impression are covered with a skin of silicone elastomer. Once the skin has cured, the resulting cover can be removed from the positive impression of the user's residual limb and is ready for use.

The principle advantage of this arrangement is that the resilient member is not removed from the positive impression of the user's residual limb until after the skin is applied, and as a consequence the chances of the resilient member changing in shape as it is manipulated are considerably reduced.

As will be appreciated by persons skilled in the art, the aforementioned method provides a bespoke residual limb cover that is customised for the particular requirements of the user. For off-the-shelf manufacturing, the covers could readily be produced—for example by means of an injection moulding process—in a variety of different combinations of residual limb size and larger/smaller region diameter differentials. Slight subsequent adjustment to fit the particular requirements of a given user could be provided by externally shaving the larger region of the cover (and to accommodate this the thickness of the skin of the larger region could be increased slightly), or by providing the user with a sock over which the cover is worn. With such an arrangement, liners according to the present invention could be manufactured relatively quickly and at relatively low unit cost.

It will be appreciated that whilst various aspects and embodiments of the present invention have heretofore been described, the scope of the present invention is not limited to the particular arrangements set out herein and instead extends to encompass all arrangements, and modifications and alterations thereto, which fall within the scope of the appended claims.

For example, whilst certain embodiments depict the resilient member as extending around and under the whole of the distal end of the cover, it will be apparent that the resilient member could simply form a circumferential ring about the periphery of the cover. In another embodiment a plurality of resilient members could be provided (for example a number of discrete resilient members arranged circumferentially about the distal end of the cover), and in another embodiment a cover with a resilient member as described herein could be provided with a supplemental deformable peripheral ring that fits into a circumferential groove in the socket to provide an additional interlock between the cover and the prosthesis.

It should also be noted that whilst the accompanying claims set out particular combinations of features described herein, the scope of the present invention is not limited to the particular combinations hereafter claimed, but instead extends to encompass any combination of features herein disclosed.

1. A prosthesis and a cover for a residual limb, wherein:
the cover comprises a first region that is normally of a greater circumference than an adjacent second region of the cover, said first region being resiliently deformable so as to temporarily reduce the circumference of that region of the cover, and
the prosthesis comprises a portion that defines an internal cavity into which a residual limb that is at least partly covered by said cover may be inserted, wherein said cavity is configured to include a first region that has a first cross-sectional area and below said first region in the direction of the limb insertion, a second region that has a second cross-sectional area larger than said first,
the arrangement being such that said resiliently deformable first region of said cover deforms to reduce the circumference of that region of said cover as the cover moves through said first region of the cavity, and expands in said second region of the cavity to thereby couple the prosthesis to the user's covered residual limb.

2. A prosthesis and a cover according to claim 1, wherein the cover comprises a nipple projecting from a distal end thereof, the prosthesis includes a complementary socket opening to said internal cavity, and the nipple is configured to deform to couple with said socket to enhance coupling of the cover to the prosthesis.

3. A prosthesis and a cover according to claim 2, wherein the nipple is integrally formed with the cover.

4. A prosthesis and a cover according to claim 2, wherein the nipple is removable from the cover.

5. A prosthesis and a cover according to claim 4, wherein the nipple is formed on a peg that is engageable with a retaining member provided in the cover.

6. A prosthesis and a cover according to claim 2, wherein the socket comprises a releasable locking mechanism that can be actuated to at least partly release the nipple from engagement with the socket.

7. A prosthesis and a cover according to claim 1, wherein the cover comprises a peg projecting from a distal end thereof, and the prosthesis includes a complementary locking member configured to releasably receive the peg.

8. A prosthesis and a cover according to claim 1, wherein the first portion includes a resilient member.

9. A prosthesis and a cover according to claim 8, wherein the resilient member comprises a gel pad.

10. A prosthesis and a cover according to claim 9, wherein the gel pad is of silicone elastomer.

11. A prosthesis and a cover according to claim 10, wherein the silicone gel paid is of 20 to 40 shore hardness, preferably from 25 to 35 shore hardness, and most preferably roughly 30 shore hardness.

12. A prosthesis and a cover according to claim 8, wherein the resilient member is in the region of 5-15 mm, preferably around 7-10 mm, thick at its widest point.

13. A prosthesis and a cover according to claim 8, wherein the cover additionally comprises a skin.

14. A prosthesis and a cover according to claim 12, wherein the skin is of silicone elastomer.

15. A prosthesis and a cover according to claim 13, wherein the skin has a shore hardness of 10 to 30, preferably a shore hardness of 10 to 20, most preferably a shore hardness of roughly 20.

16. A prosthesis and a cover according to claim 12, wherein the skin is in the region of 1 to 5 mm, preferably roughly 2 mm, thick.

17. A residual limb cover for a cooperating limb cover and prosthesis system, said prosthesis comprising a portion that defines an internal cavity into which a residual limb that is at least partly covered by said cover may be inserted, said cavity being configured to include a first region that has a first cross-sectional area and below said first region in a direction of limb insertion, a second region that has a second cross-sectional area larger than said first, said cover comprising:
a first region that is normally of a great circumference than an adjacent second region of the cover, said first region being resiliently deformable so as to temporarily reduce the circumference of that region of said cover:
the arrangement being such that said resiliently deformable first region of said cover is configured to deform to reduce the circumference of that region of said cover as the cover moves through said first region of the cavity, and to expand in said second region of the cavity to thereby couple the prosthesis to the user's covered residual limb.

18. A cover according to claim 17, wherein the cover comprises a nipple projecting from a distal end thereof, the prosthesis includes a complementary socket opening to said internal cavity, and the nipple is configured to deform to couple with said socket to enhance coupling of the cover to the prosthesis.

19. A cover according to claim 18, wherein the nipple is integrally formed with the cover.

20. A cover according to claim 19, wherein the nipple is removable from the cover.

21. A cover according to claim 10, wherein the nipple is formed on a peg that is engageable with a retaining member provided in the cover.

22. A prosthesis and a cover according to claim 17, wherein the cover comprises a peg projecting from a distal end thereof, and the prosthesis includes a complementary locking member configures to releasably receive the peg.

23. A cover according to claim 17, wherein the first portion includes a resilient member.

24. A cover according to claim 23, wherein the resilient member comprises a gel pad.

25. A cover according to claim 24, wherein the gel pad is of silicone elastomer.

26. A cover according to claim 25, wherein the silicone gel pad is of 20 to 40 shore hardness, preferably from 25 to 35 shore hardness, and most preferably roughly 30 shore softness.

27. A cover according to claim 23, wherein the resilient member is in the region of 5-15 mm, preferably around 7-10 mm, thick at its widest point.

28. A cover according to claim 23, wherein the cover additionally comprises a skin.

29. A cover according to claim 28, wherein the skin is of silicone elastomer.

30. A cover according to claim 29, wherein the skin has a shore hardness of 10 to 30, preferably a shore hardness of 10 to 20, most preferably a shore hardness of roughly 20.

31. A cover according to claim 28, wherein the skin is in the region of 1 to 5 mm, preferably roughly 2 mm, thick.

32. A prosthesis for a cooperating limb cover and prosthesis system, said cover comprising a first region that is normally of a greater circumference than an adjacent second region of the cover, said first region being resiliently deformable so as to temporarily reduce the circumference of that region of said cover; said prosthesis comprising:
a portion that defines an internal cavity into which a residual limb that is at least partly covered by said cover may be inserted, said cavity being configured to include a first region that has a first cross-sectional area and below said first region in a direction of limb insertion, a second region that has a second cross-sectional area larger than said first,
the arrangement being such that said resiliently deformable first region of said cover is configured to deform to reduce the circumference of that region of said cover as the cover moves through said first region of the cavity, and to expand in said second region of the cavity to thereby couple the prosthesis to the user's covered residual limb.

33. A prosthesis according to claim 32, wherein the cover comprises a nipple projecting from a distal end thereof, the prosthesis includes a complementary socket opening to said internal cavity, and the nipple is configured to deform to couple with said socket to enhance coupling of the cover to the prosthesis.

34. A prosthesis according to claim 32, wherein the nipple is formed on a peg that is engageable with a retaining member provided in the cover.

35. A prosthesis according to claim 33, wherein the socket comprises a releasable locking mechanism that can be actuated to at least partly release the nipple from engagement with the socket.

36. A prosthesis according to claim 32, wherein the cover comprises a peg projecting from a distal end thereof, and the prosthesis includes a complementary locking member configured to releasably receive the peg.

37. A method of making a cover for a residual limb, the method comprising:
forming a positive impression of the residual limb;
forming a resiliently deformable pad on a distal end of said positive impression;
forming a skin on said resiliently deformable pad and at least a portion of the positive impression to provide said cover.

38. A method according to claim 37 comprising the step of removing said cover from the positive impression of said residual limb.

39. A method to claim 37, wherein said skin and said resiliently deformable pad are of silicone elastomer.

40. A method according to claim 39, wherein said skin and said resiliently deformable pad are of silicone elastomer having the same or different shore hardness.
